(19) United States
(12) Patent Application Publication
(10) Pub. No.: US 2023/0021851 A1
(43) Pub. Date: Jan. 26, 2023

(54) Physiological Signal Assisted Acupuncture System and Automatic Electroacupuncture System

(71) Applicant: Wellness Allied Inc, Natick, MA (US)

(72) Inventors: Weihui Li, Natick, MA (US); Tao ZHANG, Natick, MA (US)

(21) Appl. No.: 17/383,515

(22) Filed: Jul. 23, 2021

(51) Int. Cl. A61H 39/08; A61H 39/00; A61M 21/02

(52) U.S. Cl. CPC A61H 39/086; A61H 39/002; A61M 21/02; A61H 2230/045; A61H 2230/655; A61H 2201/5007; A61H 2201/5043; A61H 2201/5097; A61H 2230/405; A61H 2230/065; A61H 2201/10; A61M 2021/0027

(57) ABSTRACT

A system that assists acupuncture based on the physiological signals of the human body. Physiological signals comprise respiratory signals, electrocardiographic signals, electrical skin signals, electroencephalograph signal, blood flow signals, pulse wave signals and/or signals derived from above signals. The acupuncture treatment process comprises the following: controlling the actuator of the automatic acupuncture device through physiological signals to start, to stop acupuncture treatment or to perform acupuncture techniques such as twisting or lifting-thrusting. It also provides an automatic acupuncture system to assist acupuncture based on Chinese Medicine Body Clock information.

### TECHNICAL FIELD

The disclosure herein relates to a system that measures physiological signals of the human body and controls an acupuncture device to do acupuncture treatment.

### BACKGROUND

Acupuncture has a history of more than 2,000 years in China. Traditional Chinese medicine believes that there is Qi and blood in the human body that regulate the basic functions of the human body. The theory of acupuncture is mainly based on this theory of Qi and blood. Acupuncture treatment is done to ensure that the human body has enough Qi and blood to circulate smoothly throughout the body. During acupuncture, an acupuncturist inserts needles (usually filiform needles) into the patient's body at a certain angle and on certain locations to treat diseases. Sometimes, an acupuncturist uses techniques such as twisting and lifting-thrusting to stimulate specific parts of the human body to enhance the acupuncture effectiveness.

To make the acupuncture treatment more effective, acupuncturists insert, remove needles or uses acupuncture lifting-thrusting or twisting techniques according to some observable physiological signals of the patient. For example, acupuncturists often insert needles when the patients inhale and remove needles when patients exhale.

Patients usually receive acupuncture when they go to hospitals or clinics during the day. However, according to the meridian theory of traditional Chinese medicine, each meridian has its own active or quiet time. In this way, the best time for treatment may be at night or early in the morning. In addition, the onset or flareup of various diseases is not limited to the daytime, such as insomnia and headaches. Therefore, the market urgently needs an automatic acupuncture device that users can operate at home anytime when needed. It is also ideal for the automatic acupuncture device to perform acupuncture treatment according to the physiological signals of the human body for better effectiveness.

### SUMMARY

The inventor previously invented an automatic acupuncture device that people can do acupuncture treatment at their home when they need [US20210022959A1]. This solves the problem of unavailable acupuncture treatment during night. The inventor further brought up the idea to integrate the measurement of physiological signals with the automatic acupuncture device to control acupuncture treatment accordingly.

According to one aspect of the invention, a physiological signal assisted acupuncture system that utilizes human physiological signals to perform acupuncture treatment, comprising: automatic acupuncture device, which insert acupuncture needles into human body, remove needles or implement acupuncture techniques; physiological signal acquisition instrument, used to measure human physiological signals; control system, which controls the acupuncture device to insert acupuncture needles, remove needles, or perform acupuncture techniques, based on the physiological signals.

Optionally, the physiological signal assisted acupuncture system further comprises a display device to display user interface for information input and output, wherein interface for information input is for users to input personal information and health conditions, or to configure the acupuncture system, and wherein the user interface for information output is to display operation guidelines and/or to instruct users for acupuncture treatments.

Optionally, the physiological signals are one or more of ECG, skin impedance, respiration, EEG, blood flow, pulse waves, heart rate variability, and derived data from above signals through methods of multiple scale entropy, intrinsic mode functions, Fourier transform, short-time Fourier transform, wavelet transform, or Hilbert-Huang transform.

Optionally, the control system, the physiological signal acquisition instrument and the automatic acupuncture device are physically integrated into one piece or are multiple pieces with wireless communication.

Optionally, the control system controls the acupuncture device to perform acupuncture techniques based on respiratory signals: the acupuncture device performs lifting-thrusting and/or twisting techniques after each inhale and/or exhale, or after a predetermined times of breathing cycles.

Optionally, the control system controls acupuncture needles of the automatic acupuncture device to move up or down a predetermined distance at a predetermined timing after the breathing signal reaches troughs of the signal, and the control system controls the acupuncture needle to move down or up a predetermined distance at a predetermined timing after breathing signal reaches peaks of the signal.

Optionally, the control system calculates a user's stress levels according to physiological signals and performs acupuncture treatment when the user is determined to be stressed.

Optionally, the control system judges whether the user is awake or asleep and/or determines sleep quality of the user based on the physiological signals: when the user wakes up in middle of night or when the sleep quality is below a predetermined threshold, the control system starts acupuncture treatments and/or performs acupuncture techniques; when the user falls asleep or the sleep quality is above a predetermined threshold, the control system stops acupuncture treatment.

Optionally, a method to determine whether the user is awake, asleep and to determine the user's sleep quality is based on cardiopulmonary coupling: high frequency coupling band of 0.1˜0.4 Hz reflects characteristics of steady-state non-rapid eye movement sleep (NREM) or stable sleep; low-frequency coupling band of 0.01˜0.10 Hz reflects characteristics of non-steady-state NREM sleep or unstable sleep; extremely low frequency coupling band of 0.001˜0.010 Hz is related to rapid eye movement phase or awake.

Optionally, the physiological signals are used to diagnose human body imbalances or diseases, and diagnosis results are based on modern medical science findings and/or diagnosis results are mapped to Chinese Medicine diagnosis from Eight-Principle Differentiation, Defense-Qi-Nutrient-Blood Differentiation, Organ Visceral Differentiation, Six Meridian Differentiation, Qi Blood Body Fluid Differentiation or Triple Warmer Differentiation.

Optionally, pulse waves are used to diagnose human body imbalances or diseases and the method is as follows: comparing the pulse wave characteristics from Cun, Guan, Chi positions of radial arteries on wrists, if one or more pulse wave characteristic values are bigger or smaller than other values with a predetermined threshold, or one or more pulse wave characteristic values are bigger or smaller than population means with a predetermined threshold, the organs that are related to that one or more positions are diagnosed to have imbalances or disorders; wherein pulse wave characteristics comprise strength of the pulse wave, spectrum powers, and/or propagation speed of the pulse waves.

Optionally, electrodermal activity is used to diagnose human body imbalances or diseases and the method is as follows: measuring and comparing skin impedances of multiple meridians, if skin impedances of one or more meridians value is higher or lower than other values with a predetermined threshold, corresponding organ(s) are diagnosed to be in imbalance state(s) and relevant acupuncture points are selected to do acupuncture treatment.

Optionally, ECG is used to diagnose human body imbalances or diseases and the method is as follows: calculating the heart rate variability from ECG signal, calculating spectral domain characteristics based on the heart rate variability, and determining human body imbalances or diseases based on power of the spectrum.

Optionally, when human body imbalances or diseases are diagnosed, the acupuncture points are selected for treatment according to Chinese Medicine Theory, or according to acupuncture effectiveness of different acupuncture points, wherein acupuncture effectiveness is manifested by improvements of patient's symptoms and/or improvements of physiological biomarkers.

Optionally, the physiological signal assisted acupuncture system further comprises a clock instrument, configured to provide time information for the control circuit to control the acupuncture device to perform acupuncture on human body at specific times.

Optionally, the control circuit controls the acupuncture device to provide acupuncture to treat imbalanced organs and/or their relevant organs at specific times according to Chinese Medicine Body Clock Theory, which describes that starting from 11:00 pm, the corresponding human organs that dominate every two hours are: gallbladder, liver, lung, large intestine, stomach, spleen, heart, small intestine, bladder, kidney, pericardium, and triple warmer.

Optionally, the relevant organs are determined by five element generating and controlling theory and/or exterior-interior correlation theory; wherein, the five element generating and controlling theory states that liver generates heart, controls spleen, is generated by kidney and is controlled by lung; heart generates spleen, controls lung, is controlled by kidney and is generated by liver; spleen generates lung, controls kidney, is controlled by liver and generated by heart; lung generates kidney, controls liver, is controlled by heart and generated spleen; kidney generates liver, controls heart, is controlled by spleen and generated by lung; wherein the exterior-interior correlation theory states that lung correlates with large intestine, heart correlates with small intestine, liver correlates with gall bladder, spleen correlates with stomach, kidney correlates with bladder, and pericardium correlates with triple warmer.

Optionally, the acupuncture device of the physiological signal assisted acupuncture system holds two or more acupuncture needles and is configured to electrically stimulate the human body when the power is turned on.

Optionally, the waveform of the electrical stimulation is sine waves, triangle waves, square waves of one or more frequencies, or density waves comprising of one or more of these waveforms.

Optionally, the physiological signal assisted acupuncture system further comprises a speaker or headphone, configured to generate a sound to stimulate or calm users, and the control circuit controlling the acupuncture device to perform acupuncture works in synergy with the sound.

Optionally, the sound is a piece of music, a piece of nature sound, and/or a piece of synthesized sound of different frequencies.

Optionally, the sound and the control signal are controlled by respiratory signals and/or work in synergy with the respiratory signals.

### BRIEF DESCRIPTION OF FIGURES

FIG. 1 shows a schematic diagram of the components and application of a human physiological signal assisted acupuncture system according to an embodiment of the present invention.

FIG. 2A-2C show a timing diagram to perform acupuncture manipulations, a schematic diagram of controlling movement of an automatic acupuncture device, and a schematic diagram showing the acupuncture needle movement direction.

FIG. 3 shows a schematic diagram demonstrating the start of acupuncture treatment when the control system determines that the human body is in a tense state based on electrodermal activity signals (EDA).

FIG. 4A shows a cardiopulmonary coupling (CPC) calculated from ECG R-R interval; FIG. 4B shows a schematic diagram of starting acupuncture or stopping acupuncture according to sleep status determined from CPC.

FIG. 5 shows a schematic diagram to diagnose organs imbalance status based on pulse waves.

FIG. 6A shows a schematic diagram of the EDA measurement device according to an embodiment of the present invention; FIG. 6B shows a column graph showing the skin impedance measured by the EDA measurement device, which can be used to diagnose patients and choose treatment plans.

FIG. 7A shows a schematic diagram of a Chinese medicine body clock. FIG. 7B shows a schematic diagram of the five elements generating and controlling relationships.

FIG. 8A shows a schematic diagram of an automatic electro-acupuncture system with two or more acupuncture needles. FIG. 8B shows a schematic diagram of electrical current pulse waves loaded on the acupuncture needle.

FIG. 9 shows a schematic diagram of an automatic acupuncture system that performs acupuncture in synergy with a sound signal that stimulates a user.

### DETAILED DESCRIPTION

The embodiments of the present invention will be described in detail below with reference to the accompanying drawings.

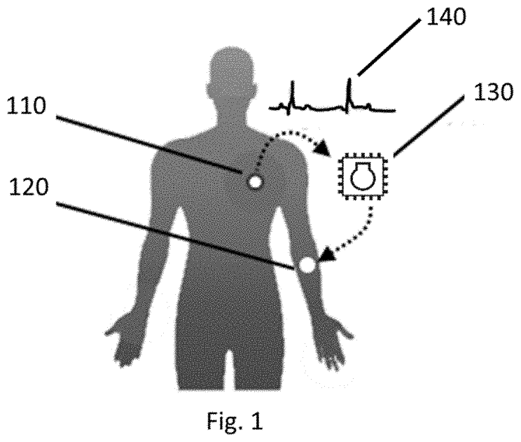

FIG. 1 shows a schematic diagram of the components and application of a human physiological signal assisted acupuncture system according to an embodiment of the present invention. The numeral 110 indicates a physiological signal acquisition device, the numeral 120 indicates an automatic acupuncture device, the numeral 130 indicates a control system. The numeral 140 indicates a physiological signal. In this example, the signal is electrocardiograph (ECG). The acupuncture system first collects physiological signals through the physiological signal acquisition device, and then performs signal processing and analysis on the physiological signals to control the automatic acupuncture device to perform acupuncture, stop acupuncture, or perform acupuncture techniques such as twisting and/or lifting-thrusting.

The physiological signal acquisition device 110 is used to collect physiological signals of the human body. The physiological signals of the human body may be one or more of electrocardiogram (ECG), skin impedance, respiration signal, electroencephalograph (EEG), blood flow, pulse wave, heart rate variability (HRV), or derived data from above signals through methods of multiple scale entropy, intrinsic mode functions, Fourier transform, short-time Fourier transform, wavelet transform, or Hilbert-Huang transform.

The automatic acupuncture device 120 can receive the control signal to automatically insert the acupuncture needle into the human body. The control signal may come from the control system 130, or may be triggered by, for example, an operator touching a button.

Based on the physiological signals of the human body, the control system 130 controls the acupuncture device to start acupuncture, stop the acupuncture, or perform acupuncture techniques such as lifting-thrusting and/or twisting to enhance acupuncture effects.

For example, the control system can control the acupuncture device to perform acupuncture on human tissue based on the respiratory signal. Specifically, the acupuncture system may perform the acupuncture technique of lifting-thrusting and/or twisting every time the patient inhales and/or exhales, or after every a few predetermined breathing cycles.

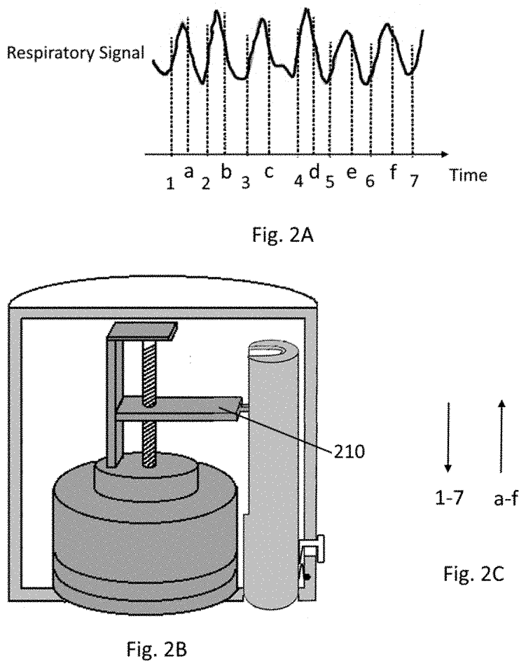

FIG. 2A shows a schematic diagram of the timing that the acupuncture device performs acupuncture manipulations according to breathing signals. Specifically, the control system controls the automatic acupuncture device to perform acupuncture manipulations such as lifting-thrusting and twisting at a specific time in each breathing cycle. The technique of lifting-thrusting and twisting lasts for a predetermined length of time. The specific moment may start at a certain time after the lowest point of respiration or after a peak of a respiration signal. FIG. 2C shows the moving directions of the acupuncture needle at timing 1-7 and a-f. At timing 1-7, the control system controls the acupuncture needle driver 210 in FIG. 2B to move down a specific distance. At timing a-f, the control system controls the acupuncture needle driver 210 in FIG. 2B to move upward a certain distance. Inserting the needle, removing needle, or performing acupuncture techniques according to the breath can nourish human body if the body is in a deficiency, or deplete body if the body is in an excess state. In the traditional Chinese Medicine classic book “Su Wen—Li he Zhen xie Lun”, it states: inserting needle during inhalation won't be against Qi flow; allowing needle to stay quietly for a moment won't disturb the Qi; twisting the needle during inhalation to catch Qi, removing needle during exhalation.

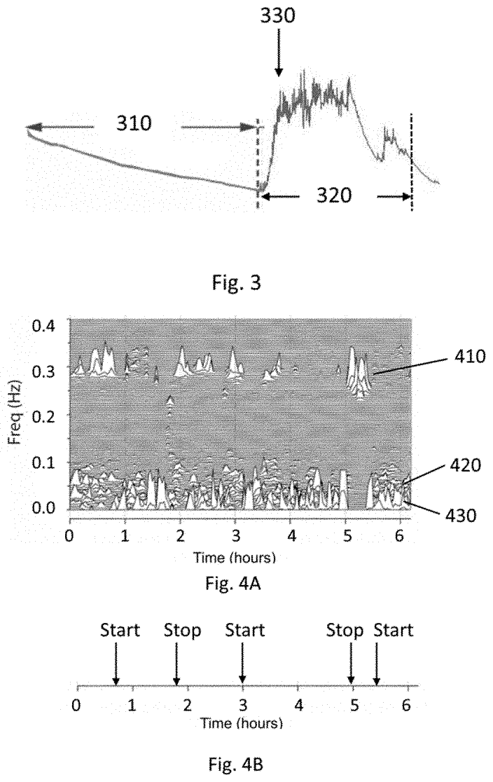

FIG. 3 shows a schematic diagram of determining acupuncture timing by monitoring electrodermal activities according to an embodiment of the present invention. Smooth segment 310 indicates that the body is in a relaxed status. Segment 320 has high frequency data and indicates the body enters a stress condition. 330 shows that the acupuncture device detects the stress and starts the acupuncture treatment. Acupuncture will be able to relieve the body stress.

FIGS. 4A and 4B show schematic diagrams of determining acupuncture timing by sleep quality according to an embodiment of the present invention. FIG. 4A shows the cardiopulmonary coupling (CPC) calculated from an ECG RR interval. For details, refer to the paper by Robert Thomas et al [1]. The peak values of 410, 420 and 430 indicate the coherent crossover power of the high frequency of 0.1-0.4 Hz, low frequency of 0.01-0.1 Hz and extremely low frequency of 0.001˜0.010 Hz of the RR interval. 410 indicates good sleep quality, 420 indicates poor sleep quality and 430 indicates rapid eye movement phase or awake.

FIG. 4B shows a schematic diagram of starting or stopping acupuncture treatment with the change of sleep quality according to an embodiment of the present invention. FIGS. 4A and 4B share the same X axis. When the sleep quality is determined to be worse with high frequency coherent crossover power being smaller and low frequency/extremely low frequency coherent crossover power being larger, the acupuncture device starts the acupuncture treatment. When the sleep quality is determined to be better with higher frequency coherent crossover power being larger and low frequency/extremely low frequency coherent crossover power being smaller, the acupuncture device stops acupuncture. By automatically starting or stopping acupuncture treatment according to sleep status, it is possible to reduce the dependence on the operator, and further improve the effect of acupuncture treatment.

FIG. 5 shows a schematic diagram of organ imbalance diagnosis through the strength of the pulse waves according to an embodiment of the present invention. The pulses measured at positions on the left wrist 510 (Chi), 520 (Guan), 530 (Cun) represent kidney yin, liver, and heart, and the pulses measured at positions on the right wrist 540 (Cun), 550 (Guan), 560 (Chi) represent the lung, spleen, and kidney yang. Waveforms 515, 525, 535, 545, 555, 565 show the corresponding pulse waves measured at 510, 520, 530, 540, 550, and 560. According to an embodiment of the present invention, a method for organ imbalance diagnosis and acupuncture treatment is: comparing the signal characteristics of the Cun, Guan, Chi pulse waves of the left and right hands (signal characteristics comprise the strength of the pulse wave, the frequency spectrum of the pulse wave, and the speed of the pulse wave), determining the organ that is out of balance, and selecting the relevant acupuncture points accordingly for acupuncture treatment. For example, one way to determine the imbalance of lung function is: the strength of the pulse wave in the position 540 is weaker or stronger than the strength of the pulse waves in other parts. In the figure, the pulse wave 545 is weaker than other pulse waves, thus we can determine that the lung is out of balance. After determining the imbalance of lung function, the acupuncture device selects the corresponding acupuncture points.

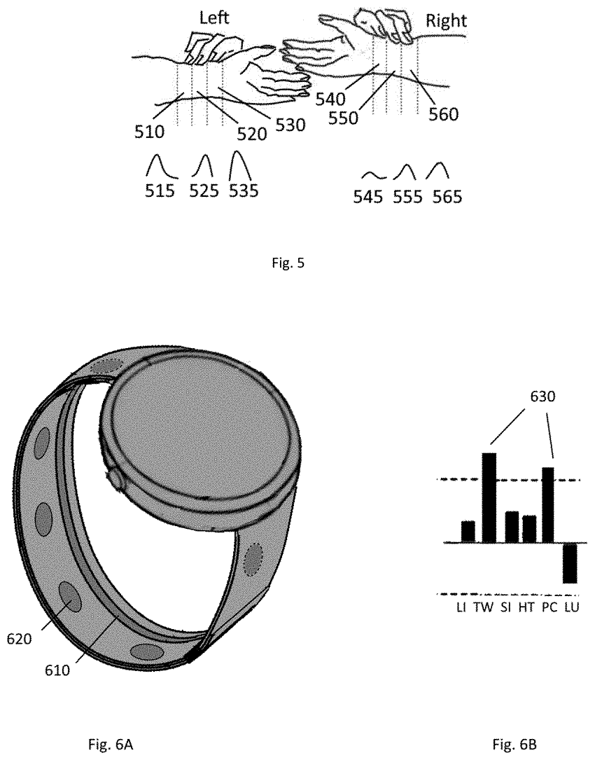

FIG. 6A-6B show schematic diagrams of acupuncture performed by an acupuncture device combined with an electrodermal activity measurement device according to an embodiment of the present invention. FIG. 6A is an example of an electrodermal activity measurement device in the form of a watch. 610 is a common reference electrode and 620 is one of several measurement electrodes. FIG. 6B schematically shows the values of six meridians. 630 shows that the values of the two meridians (Triple warmer or TW and Pericardium or PC) are higher than the threshold. Therefore, TW and PC are determined to be out of balance, and the acupuncture device performs acupuncture treatment on the corresponding acupuncture points based on this diagnosis.

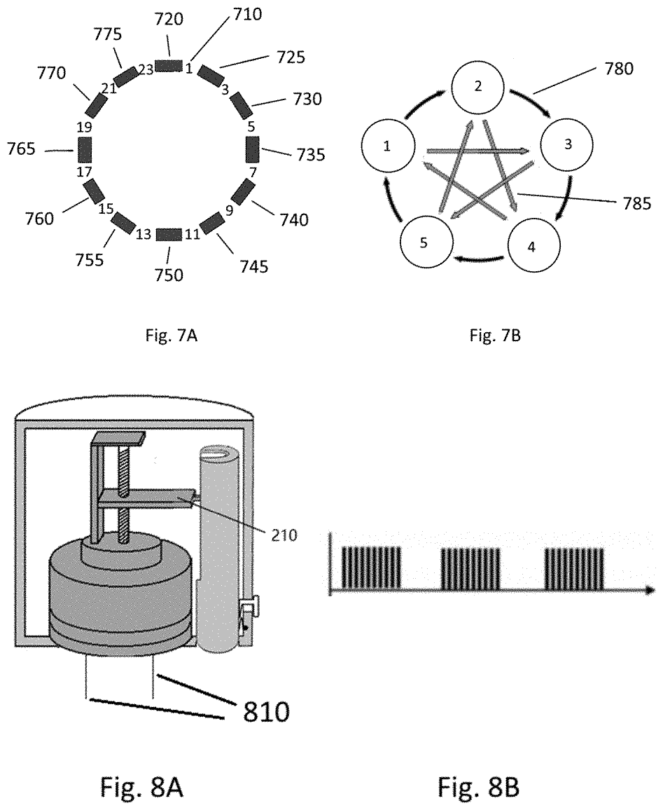

FIGS. 7A and 7B show diagrams of determining the timing of acupuncture based on the Chinese Medicine body clock and the five-element generating/controlling diagram. FIG. 7A shows the schematic diagram of Chinese medicine body clock. 710 represents 1 o'clock in the morning, and the other numbers represent 3 o'clock, 5 o'clock, 7 o'clock, 9 o'clock, 11 o'clock, 13 o'clock, 15 o'clock, 17 o'clock, 19 o'clock, 21 o'clock and 23 o'clock in turns. 720, 725, 730, 735, 740, 745, 750, 755, 760, 765, 770, 775 represent the time intervals dominated by gallbladder, liver, lung, large intestine, stomach, spleen, heart, small intestine, bladder, kidney, pericardium, and triple warmer, respectively. To determine the timing to do acupuncture, the system needs to find out which organ is out of balance. For example, when the system determines that the heart is out of balance, the acupuncture device starts acupuncture treatment at 11 o'clock. FIG. 7B shows a schematic diagram of five element generating/controlling diagram. 1 is wood, representing liver, 2 is fire, representing heart, 3 is earth, representing spleen, 4 is metal, representing lung, and 5 is water, representing kidney. 780 indicates the generating relationship, and 785 represents the controlling relationship. When the heart is out of balance, we can treat the organ that is generated from the heart—the spleen, the organ that generates heart—the liver, the organ that is controlled by heart—the lung, the organ that controls heart—the kidney. According to one embodiment of the present invention, an automatic acupuncture system that can treat disorders according to the Chinese medicine body clock is provided, comprising the following: a clock information providing clock information; an acupuncture device; and a control circuit that controls the acupuncture device by timing or by human physiological signals. The acupuncture system can perform acupuncture treatment at a specific time. The specific time information is determined by the Chinese medicine body clock, for example, which states that, starting from 11:00 pm, every two hours are dominated by an organ or a part of the body, which are the gallbladder, liver, lung, large intestine, stomach, spleen, heart, small intestine, bladder, kidney, pericardium, and triple warmer respectively. Preferably, the automatic acupuncture system diagnoses one or more imbalanced organs according to the physiological signals, and then performs acupuncture treatment to treat relevant organs or body parts at a specific time according to body clock information. Relevant organs or body parts can be determined by the five elements generating/controlling relationship or by external-internal coupling relationship. The relevant organ is the organ that is generated by the imbalanced organ, the organ that generates the imbalanced organ, the organ that is controlled by the imbalanced organ, or the organ that is controlling the imbalanced organ, or the organ that is coupled with the imbalanced organ through the external-internal coupling relationship. In Chinese medicine, the external-internal correlation relationship includes the following: the liver correlates with gallbladder, kidney correlates with bladder, spleen correlates with stomach, lung correlates with large intestine, heart correlates with small intestine, and triple warmer correlates with pericardium.

FIG. 8A shows an automatic electro-acupuncture system. The acupuncture device has two or more acupuncture needles, and FIG. 8B shows a schematic diagram of the acupuncture needles loaded with a density wave current signal of a certain frequency. The current signal can also have other waveforms, for example, the waveform of the electrical stimulation is a sine wave, a triangle wave, a square wave with one or more frequencies, or a density wave composed of these waveforms. Different waveforms have little difference in therapeutic effects, but the degree of comfort for people varies with different waveforms.

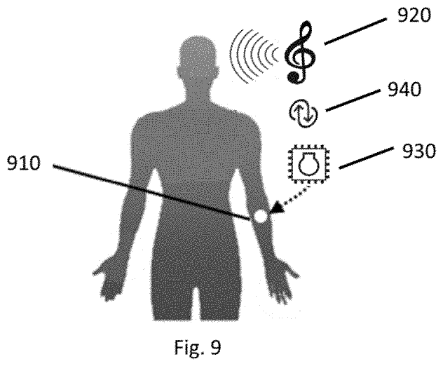

FIG. 9 shows an automatic acupuncture system that performs acupuncture in synergy with a sound that stimulates the user. 910 is the acupuncture device, 920 is a piece of sound, 930 is the control system, 940 represents that the sound and the control signal work in a synergic way to enhance the acupuncture treatment effectiveness. The sound can be a piece of music, a piece of recorded nature sound, like ocean wave tide, raining sound, birds chipping, etc. or a piece of synthesized sound of different frequencies.

Besides the sound and the control signal that are in synergy, the system can also integrate in the breathing signal and makes the sound, the control signal and the breathing signal all in synergy. This will further enhance the acupuncture effect.

In the example of FIG. 1, the physiological signal acquisition device 110, the automatic acupuncture device 120 and the control system 130 are separated, and the three units conduct information transmission through wired communication or wireless communication. Preferably, two or three of them can also be integrated together. For example, the automatic acupuncture device and the control system 130 are integrated together. Or for example, the automatic acupuncture device itself is equipped with a controller that incorporates the functions of the control system 130.

In an example, the human body physiological signal assisting acupuncture system also comprises a display device that operates to display a user input information interface and a system information display interface, where the user input information interface is for the user to input personal information and disease information, or to set up the automatic acupuncture system. The system information display interface is used to display system information to guide users to use and to provide treatment guidance.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A physiological signal assisted acupuncture system that utilizes human physiological signals to perform acupuncture treatment, comprising:
automatic acupuncture device, which insert acupuncture needles into human body, remove needles or implement acupuncture techniques;
physiological signal acquisition instrument, used to measure human physiological signals;
control system, which controls the acupuncture device to insert acupuncture needles, remove needles, or perform acupuncture techniques, based on the physiological signals.

2. The physiological signal assisted acupuncture system according to claim 1, further comprising a display device to display user interface for information input and output, wherein interface for information input is for users to input personal information and health conditions, or to configure the acupuncture system, and wherein the user interface for information output is to display operation guidelines and/or to instruct users for acupuncture treatments.

3. The physiological signal assisted acupuncture system according to claim 1, wherein the physiological signals are one or more of ECG, skin impedance, respiration, EEG, blood flow, pulse waves, heart rate variability, and derived data from above signals through methods of multiple scale entropy, intrinsic mode functions, Fourier transform, short-time Fourier transform, wavelet transform, or Hilbert-Huang transform.

4. The physiological signal assisted acupuncture system according to claim 1, wherein the control system, the physiological signal acquisition instrument and the automatic acupuncture device are physically integrated into one piece or are multiple pieces with wireless communication.

5. The physiological signal assisted acupuncture system according to claim 1, wherein the control system controls the acupuncture device to perform acupuncture techniques based on respiratory signals: the acupuncture device performs lifting-thrusting and/or twisting techniques after each inhale and/or exhale, or after a predetermined times of breathing cycles.

6. The physiological signal assisted acupuncture system according to claim 5, the control system controls acupuncture needles of the automatic acupuncture device to move up or down a predetermined distance at a predetermined timing after the breathing signal reaches troughs of the signal, and the control system controls the acupuncture needle to move down or up a predetermined distance at a predetermined timing after breathing signal reaches peaks of the signal.

7. The physiological signal assisted acupuncture system according to claim 1, wherein the control system calculates a user's stress levels according to physiological signals and performs acupuncture treatment when the user is determined to be stressed.

8. The physiological signal assisted acupuncture system according to claim 1, wherein the control system judges whether the user is awake or asleep and/or determines sleep quality of the user based on the physiological signals: when the user wakes up in middle of night or when the sleep quality is below a predetermined threshold, the control system starts acupuncture treatments and/or performs acupuncture techniques; when the user falls asleep or the sleep quality is above a predetermined threshold, the control system stops acupuncture treatment.

9. The physiological signal assisted acupuncture system according to claim 8, wherein a method to determine whether the user is awake, asleep and to determine the user's sleep quality is based on cardiopulmonary coupling: high frequency coupling band of 0.1˜0.4 Hz reflects characteristics of steady-state non-rapid eye movement sleep (NREM) or stable sleep; low-frequency coupling band of 0.01˜0.10 Hz reflects characteristics of non-steady-state NREM sleep or unstable sleep; extremely low frequency coupling band of 0.001˜0.010 Hz is related to rapid eye movement phase or awake.

10. The physiological signal assisted acupuncture system according to claim 1, wherein the physiological signals are used to diagnose human body imbalances or diseases, and diagnosis results are based on modern medical science findings and/or diagnosis results are mapped to Chinese Medicine diagnosis from Eight-Principle Differentiation, Defense-Qi-Nutrient-Blood Differentiation, Organ Visceral Differentiation, Six Meridian Differentiation, Qi Blood Body Fluid Differentiation or Triple Warmer Differentiation.

11. The physiological signal assisted acupuncture system according to claim 10, wherein pulse waves are used to diagnose human body imbalances or diseases and the method is as follows: comparing the pulse wave characteristics from Cun, Guan, Chi positions of radial arteries on wrists, if one or more pulse wave characteristic values are bigger or smaller than other values with a predetermined threshold, or one or more pulse wave characteristic values are bigger or smaller than population means with a predetermined threshold, the organs that are related to that one or more positions are diagnosed to have imbalances or disorders; wherein pulse wave characteristics comprise strength of the pulse wave, spectrum powers, and/or propagation speed of the pulse waves.

12. The physiological signal assisted acupuncture system according to claim 10, wherein electrodermal activity is used to diagnose human body imbalances or diseases and the method is as follows: measuring and comparing skin impedances of multiple meridians, if skin impedances of one or more meridians value is higher or lower than other values with a predetermined threshold, corresponding organ(s) are diagnosed to be in imbalance state(s) and relevant acupuncture points are selected to do acupuncture treatment.

13. The physiological signal assisted acupuncture system according to claim 10, wherein ECG is used to diagnose human body imbalances or diseases and the method is as follows: calculating the heart rate variability from ECG signal, calculating spectral domain characteristics based on the heart rate variability, and determining human body imbalances or diseases based on power of the spectrum.

14. The physiological signal assisted acupuncture system according to claim 10, when human body imbalances or diseases are diagnosed, the acupuncture points are selected for treatment according to Chinese Medicine Theory, or according to acupuncture effectiveness of different acupuncture points, wherein acupuncture effectiveness is manifested by improvements of patient's symptoms and/or improvements of physiological biomarkers.

15. The physiological signal assisted acupuncture system according to claim 1, further comprising a clock instrument, configured to provide time information for the control circuit to control the acupuncture device to perform acupuncture on human body at specific times.

16. The physiological signal assisted acupuncture system according to claim 15, wherein the control circuit controls the acupuncture device to provide acupuncture to treat imbalanced organs and/or their relevant organs at specific times according to Chinese Medicine Body Clock Theory, which describes that starting from 11:00 pm, the corresponding human organs that dominate every two hours are: gallbladder, liver, lung, large intestine, stomach, spleen, heart, small intestine, bladder, kidney, pericardium, and triple warmer.

17. The physiological signal assisted acupuncture system according to claim 16, wherein the relevant organs are determined by five element generating and controlling theory and/or exterior-interior correlation theory; wherein, the five element generating and controlling theory states that liver generates heart, controls spleen, is generated by kidney and is controlled by lung; heart generates spleen, controls lung, is controlled by kidney and is generated by liver; spleen generates lung, controls kidney, is controlled by liver and generated by heart; lung generates kidney, controls liver, is controlled by heart and generated spleen; kidney generates liver, controls heart, is controlled by spleen and generated by lung; wherein the exterior-interior correlation theory states that lung correlates with large intestine, heart correlates with small intestine, liver correlates with gall bladder, spleen correlates with stomach, kidney correlates with bladder, and pericardium correlates with triple warmer.

18. The physiological signal assisted acupuncture system according to claim 1, wherein the acupuncture device holds two or more acupuncture needles and is configured to electrically stimulate the human body when the power is turned on.

19. The physiological signal assisted acupuncture system according to claim 18, wherein the waveform of the electrical stimulation is sine waves, triangle waves, square waves of one or more frequencies, or density waves comprising of one or more of these waveforms.

20. The physiological signal assisted acupuncture system according to claim 1 further comprising a speaker or headphone, configured to generate a sound to stimulate or calm users, and the control circuit controlling the acupuncture device to perform acupuncture working in synergy with the sound.

21. The physiological signal assisted acupuncture system according to claim 20, wherein the sound is a piece of music, a piece of nature sound, and/or a piece of synthesized sound of different frequencies.

22. The physiological signal assisted acupuncture system according to claim 20, wherein the sound and the control signal are controlled by respiratory signals and/or work in synergy with the respiratory signals.
